(19) United States
(12) Patent Application Publication
(10) Pub. No.: US 2005/0227885 A1
(43) Pub. Date: Oct. 13, 2005

(54) Soap bar

(76) Inventor: Rafael Maric, Andrews (AU)

Correspondence Address: SUGHRUE MION, PLLC, 2100 PENNSYLVANIA AVENUE, N.W., SUITE 800, WASHINGTON, DC, 20037, US

(21) Appl. No.: 10/504,465

(22) PCT Filed: Feb. 13, 2002
(86) PCT No.: PCT/AU02/00146

§ 371 Date: Nov. 12, 2004

(30) Foreign Application Priority Data
Mar. 29, 2001 (AU) PR 4085
Dec. 14, 2001 (AU) PR 9536

(51) Int. Cl. A61K 7/50

(52) U.S. Cl. 510/141

(57) ABSTRACT

Provided is a soap bar (10) comprising a body (12) having a first member (14) and a second member (16) arranged to diverge from the first member (14). At least one of the first and second members (14,16) is formed of a soap material. The first and second members (14,16) are configured so that when the body is held by a person's hand said one of the first and second members (14,16) extends from the hand so that said one member can be applied for washing body parts. Said other of the first and second members (14,16) is configured so that it can be grasped by certain fingers against the palm of the person's hand while applying said one of the first and second members for washing the body parts. The configuration of the first and second members allows the soap bar of the present invention to be securely held even in the wet condition. This helps in reducing the tendency of the bar to slip out of the hand. At least said one of the first and second members is advantageously shaped so that it has a relatively narrow width at its free end and the width increases progressively towards its opposite end.

### TECHNICAL FIELD OF THE INVENTION

THIS INVENTION relates to an improved soap bar for use in washing body parts.

### BACKGROUND OF THE INVENTION

Soap bars known to the applicant are generally in a substantially rectangular shaped block and are sized for grasping wholly in a hand. In use, one grasps a soap bar with one hand and applies it to body parts that need washing. Due to its shape and size, fingers or part of the palm of the hand grasping the soap bar will come in contact with the body parts during washing.

Touching one's private body parts is unacceptable to believers of certain religions such as islam. For these believers it is extremely difficult to avoid contacting their private parts when washing with a bar soap.

People who need to wash young children or older people who cannot wash themselves also have difficulty in avoiding contact with body parts when washing with a soap bar.

The known soap bars also can not reach small crevices and gaps of the human body such as the area between buttocks, between fingers, between toes, in or behind ears, etc.

Persons with long finger nails can cause injury when applying known soap bars to body parts during washing. Contacting body parts while washing may also spread bacteria from fingers to the body parts.

Soap bars are slippery when wet and they tend to slip out of the hands onto the floors during use. Old or frail persons, and certain disabled persons can fall when attempting to pick them up.

### OBJECT OF THE INVENTION

An object of the present invention is to provide a soap bar which alleviates or reduces to a certain level one or more of the above prior art disadvantages.

### SUMMARY OF THE INVENTION

In one aspect, the invention resides in a soap bar comprising a body having a first member and a second member arranged to diverge from the first member wherein an angle of divergence between respective longitudinal axes of the first and second members is in the range of 45° to 170° and the soap bar is dimensioned for one of the member to be grasped by a person's hand while the other member extends from the hand.

The soap bar may have the first and second members formed integrally or they may have mating end surfaces that are joined together. At least one of the first and second members may be configured so that it can be urged by at least one finger against the palm of a person's hand while applying the other of the first and second members for washing the person's body. At least one of the first and second members may be configured so that when the member is grasped by a person with the person's thumb on one face at least one of the middle finger and the ring finger of the person can rest on the opposite face thereof. The soap bar may be configured to permit the person's forefinger to extend over the member extending from the person's hand to press the body into the person's palm.

At least one of the first and second members may be shaped with a relatively narrow width at its free end and with the width progressively increasing towards an opposite end of the member. Both the first and second members may be shaped with a relatively narrow width at the free end and progressively increasing width towards the opposite end of each respective member. At least one of the first and second members may have an oval or half oval shaped cross section.

The oval or half oval shaped cross section may have a large section and an opposite narrow section. The narrow section may be sized so that it can be used to wash relatively small crevices and gaps in the human body.

The body of the soap bar may have a non-slip enhancing means to resist slipping of the body from the person's hand. The non-slip enhancing means may be a substantially flat surface or a series of ridges for the forefinger or any other finger or fingers to rest upon.

Each of the first and second members may have a first side section and a second side section opposite to the first side section, and the non-slip enhancing means may include a relatively flat region on the first side section of at least one of the first and second members so that a finger of a user of the soap bar can rest on the flat region while using the soap bar.

The non-slip enhancing means may include a further relatively flat region on the second side section of at least one of the first and second members so that a finger of the user of the soap bar can rest on the further flat region while using the soap bar.

The first and second members may be arranged to diverge outwardly from a mid point of the soap bar, and the flat region and/or the further flat region may extend from the mid point along the first member and/or the second member. The angle of divergence of the soap bar may be approximately 145°. The soap bar may be configured substantially as a boomerang shape. At least one face of the soap bar may be convex. Both faces of the soap bar may be convex.

In one aspect therefore the present invention resides in a soap bar comprising a body having a first member and a second member arrange to diverge from the first member. At least one of the first and second members is formed of a soap material. The first and second members are configured so that when the body is held by a person's hand said one of the first and second members extends from the hand so that said one member can be applied for washing body parts.

The first and second members are preferably formed integrally. Alternatively they may have mating end surfaces that can be joined together.

The other of said first and second members may be formed of a soap material or a non soap material.

In preference, said other of the first and second members is configured so that it can be grasped by certain fingers against the palm of the person's hand while applying said one of the first and second members for washing the body parts. More preferably the first and second members are configured so that said other member is grasped with the thumb on one face thereof and with at least one of the middle finger and the ring finger on the opposite face thereof. It is further preferred that the forefinger extends over the one member to press the body into the palm.

The configuration of the first and second members allows the soap bar of the present invention to be securely held even in the wet condition. This helps in reducing the tendency of the bar to slip out of the hand.

The angle of divergence between the first and second members can be any angle within the range of 45° to 170°. In one specific form the angle of divergence is 145°.

At least said one of the first and second members is advantageously shaped so that it has a relatively narrow width at its free end and the width increases progressively towards its opposite end. Typically both said first and second members are shaped so that each of them has a relatively narrow width at its free end and the width increases progressively towards its opposite end. In an example the body is shaped substantially in the form of a shallow V. In another example the body is shaped substantially in the form of a boomerang.

It is further preferred that at least said one of the first and second members has an oval or half oval shaped cross section. Desirably said oval or half oval shaped cross section has a large section and an opposite narrow section. The narrow section is sized so that it can be used to wash certain crevices and gaps in the human body.

Desirably, the soap bar has means for enhancing non-slip of the forefinger. The enhancing means may be a substantially flat surface or ridges for the forefinger to rest thereon.

In preference, each of the first and second members have a first side section and a second side section opposite to the first side section. The enhancing means includes a relatively flat region on said first section of at least one of said first and second members so that a finger of a user of the soap bar can rest on the flat region while using the soap bar.

Preferably, the enhancing means includes a further relatively flat region on said second side section of at least one of said first and second members so that a finger of a user of the soap bar can rest on the further flat region while using the soap bar.

More preferably, said first and second members are arranged to diverge outwardly from the mid point of the soap bar, and said flat region and/or said further flat region extends from said mid point along the first member and/or the second member.

### BRIEF DESCRIPTION OR THE DRAWINGS

In order that the present invention can be more readily understood and be put into practical effect reference will now be made to the accompanying drawings which illustrate embodiments of the present invention and wherein:—

FIG. 1 is a schematic plan view of an embodiment of the soap bar according to the present invention;

FIG. 2 is a bottom view of the soap bar shown in FIG. 1;

FIG. 3 is a side view of the soap bar shown in FIG. 1;

FIG. 4 is an end view of the soap bar shown in FIG. 1;

FIG. 5 is a schematic plan view of another embodiment of the soap bar according to the present invention;

FIG. 6 is a bottom view of the soap bar shown in FIG. 5;

FIG. 7 is a side view of the soap bar shown in FIG. 5;

FIG. 8 is an end view of the soap bar shown in FIG. 5;

FIG. 9 is a drawing illustrating use of the soap bar shown in FIGS. 1 to 4.

FIG. 10 is a schematic top view of an embodiment of the soap bar according to the present invention;

FIG. 11 is a side view of the soap bar shown in FIG. 10;

FIG. 12 is a plan view of the soap bar shown in FIG. 10; and

FIG. 13 is a plan view of a further embodiment of the soap bar according to the present invention.

### DETAILED DESCRIPTION OF THE INVENTION

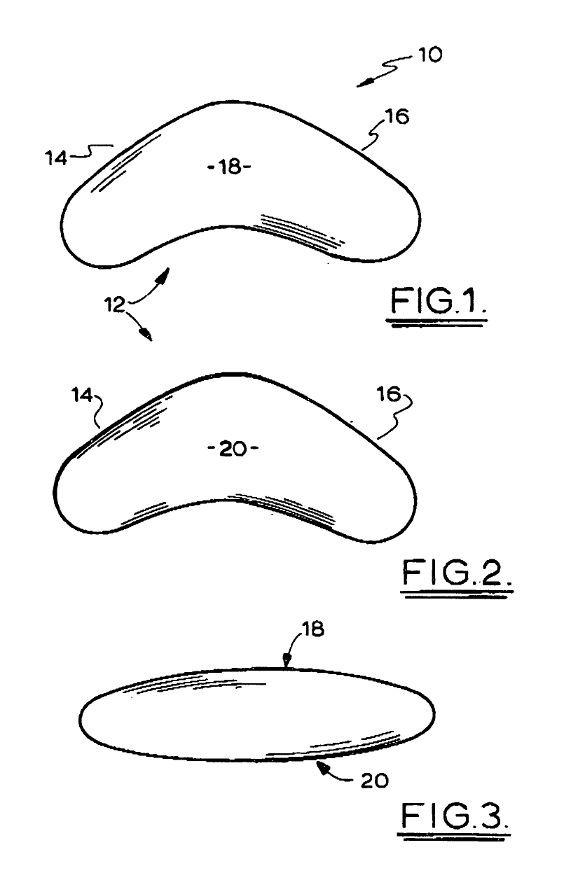

Referring to the Figures and initially to FIGS. 1 and 2 there is shown an embodiment of the soap bar 10 according to the present invention. The soap bar 10 comprises a body 10 having a first member 14 and a second member 16 formed integrally with the first member 14. The members 14 and 16 form a top face 18 and a bottom face 20, and as can be seen in the drawing the members 14 and 16 diverge from each other at an angle of about 145°. As such the body 12 is substantially boomerang shaped.

The members 14 and 16 have a width that increases progressively from their respective free ends towards the middle of the body 12. Turning to FIG. 3, it can be seen that the body 12 also has a thickness that increases progresses from their respective ends towards the middle.

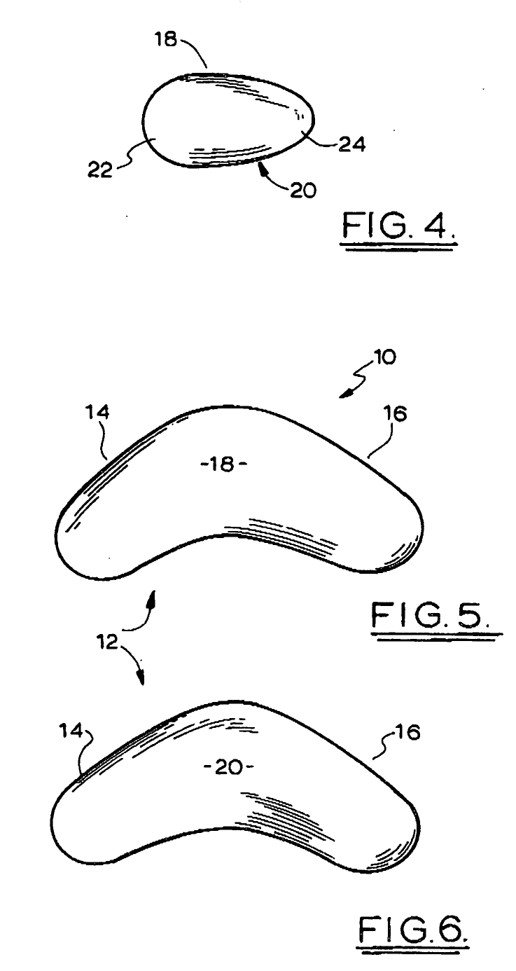

The end view of FIG. 4 shows that the body 12 has a large curved side section 22 and a narrow curved side section 24. The section 24 is sized so that it can reach small crevices, the areas behind ears, the gaps between fingers or toes, in or under genital, etc. of the human body.

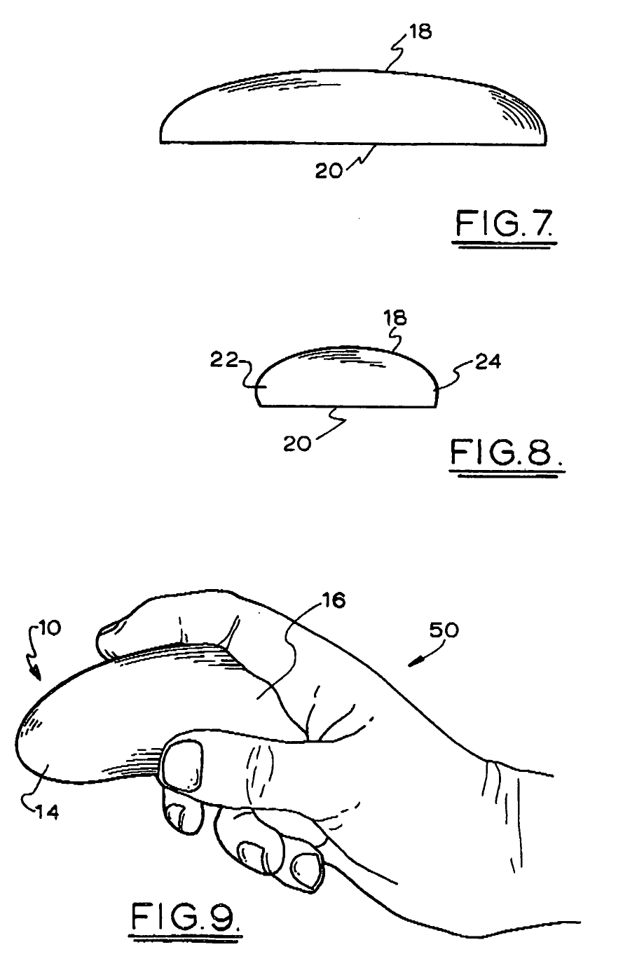

The embodiment of the soap bar 10 shown in FIGS. 5 to 8 is substantially similar to the embodiment described above. The only difference is that this embodiment has a substantially flat bottom face 20 as shown clearly in FIGS. 7 and 8.

In FIG. 9 there is shown the soap bar 10 according to the present invention being held by a hand 50 of a person. The soap bar 10 is grasped in the hand 50 with the thumb on the top face 18, the middle and the ring fingers on the bottom face 20 of the member 16, and the forefinger extending over and onto the side section 22 of the member 14.

The soap bar 10 can thus be grasped firmly even when the bar 10 is wet. It also allows a user to easily control movement and application of member 14 of the bar 10 over any part of the user's or other's body without contacting the body parts.

Either the large side section 22 or the narrow side section 24 can be used to wash the body parts. The narrow section 24 is specially sized to wash small crevices and gaps in the human body.

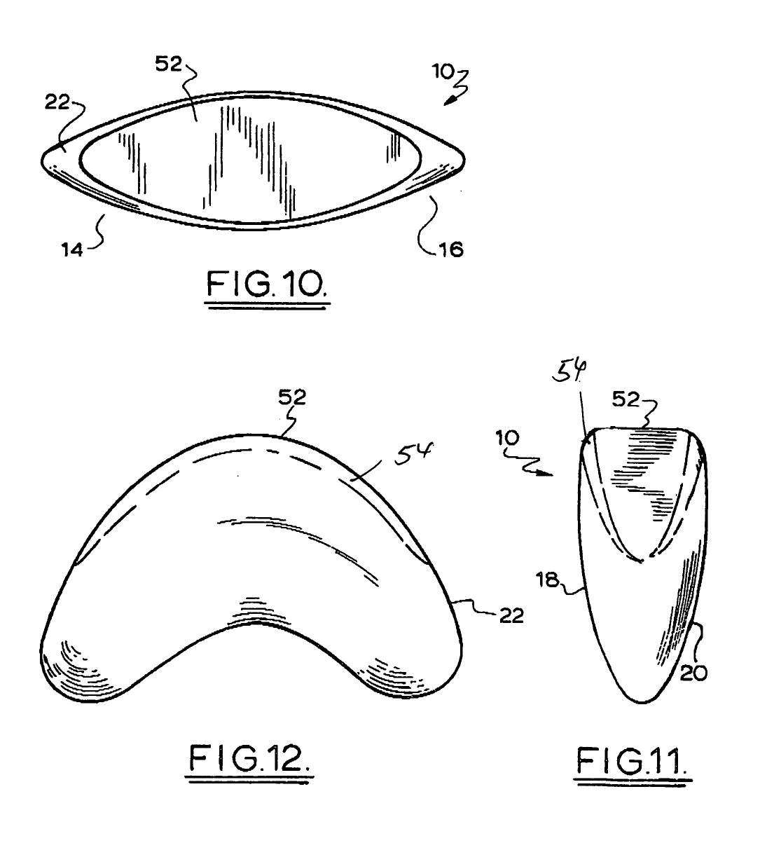

Referring to FIGS. 10 and 11 there is shown a soap bar 10 having body 12 with a first member 14 and second member 16, and a first side section 22 and a second side section 24 as shown.

FIG. 10 is a top view of the soap bar 10 and it shows a relative flat region 52 on the first side section 22. The flat region 52 extending into the first member 14 and the second member 16, from the mid point 54 (see FIG. 11) of the body 12.

FIG. 11 shows a side view of the soap bar 10 shown in FIG. 10. As can be seen, the flat region 52 extends from the mid point 54 partly along the first member 14. While not shown, it should be understood that the flat region 52 also extends partly along the second member 16.

In use, a user can grasp the soap bar 10 and rest the forefinger on the flat region 52. Accordingly, the forefinger is on a relatively flat surface and this minimises the tendency for a soap bar to slip from a user's hand.

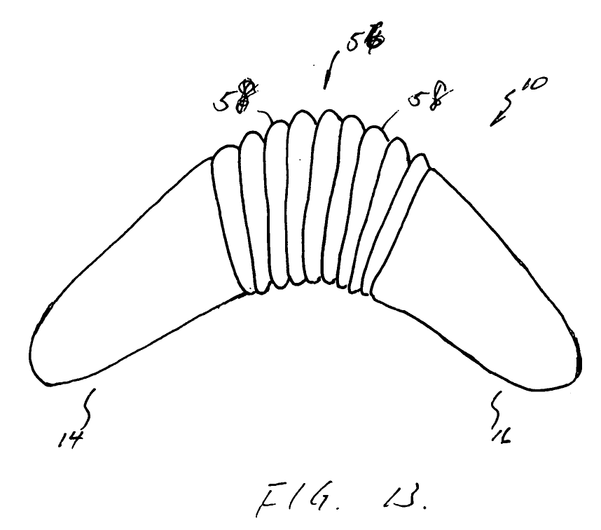

The soap bar 10 as shown in FIG. 12 has cambered corners 54 between each of the faces 18 and 20, and the flat region 52. The cambered corners 54 allow a user's fingers to bent therearound and rests comfortably on one of the corners 54.

In FIG. 13, the soap bar 10 has a substantially ridged region 546 extending from the mid point 52 into the first and second members 14 and 16. The ridges 58 on the ridged area 56 allow a user to positively grip onto the soap bar 10 and thereby enhancing the non-slip gripping effect provided by the soap bar of the invention.

Whilst the above has been given by way of illustrative example of the present invention many variations and modifications thereto will be apparent to those skilled in the art without departing from the broad ambit and scope of the invention as herein set forth.

1. A soap bar comprising a body having a first member; and
a second member arrange to diverge from the first member;
wherein:
an angle of divergence between respective longitudinal axes of the first and second members is in the range of 45° to 170° and the soap bar is dimensioned for one of the members to be grasped by a person's hand while the other member extends from the hand.

2. The soap bar according to claim 1 wherein the first and second members are formed integrally or have mating end surfaces that are joined together.

3. The soap bar according to claim 1 wherein at least one of the first and second members is configured so that it can be urged by at least one finger against the palm of a person's hand while applying the other of the first and second members for washing the person's body.

4. The soap bar according to claim 1 to wherein at least one of the first and second members are configured so that when said member is grasped by a person with the person's thumb on one. face thereof at least one of the middle finger and the ring finger of the person can rest on the opposite face thereof.

5. The soap bar according to claim 4 configured to permit the person's forefinger to extend over the member extending from the person's hand to press the body into the person's palm.

6. The soap bar according to claim 1 wherein at least one of the first and second members is shaped with a relatively narrow width at its free end, said width progressively increasing towards an opposite end of the member.

7. The soap bar according to claim 6 wherein both the first and second members are shaped with a relatively narrow width at the free end and progressively increasing width towards the opposite end of each respective member.

8. The soap bar according to claim 8 wherein at least one of the first and second members has an oval or half oval shaped cross section.

9. The soap bar according to claim 8 wherein the oval or half oval shaped cross section has a large section and an opposite narrow section, and the narrow section is sized so that it can be used to wash relatively small crevices and gaps in the human body.

10. The soap bar according to claim 1 wherein the body has non-slip enhancing means to resist slipping of the body from the person's hand.

11. The soap bar according to claim 10 wherein the non-slip enhancing means is a substantially flat surface or a series of ridges for the forefinger or any other finger or fingers to rest thereon.

12. The soap bar according to claim 11 wherein each of the first and second members has a first side section and a second side section opposite to the first side section, and the non-slip enhancing means includes a relatively flat region on the first side section of at least one of the first and second members so that a finger of a user of the soap bar can rest on the flat region while using the soap bar.

13. The soap bar according to claim 12 wherein the non-slip enhancing means includes a further relatively flat region on the second side section of at least one of the first and second members so that a finger of the user of the soap bar can rest on the further flat region while using the soap bar.

14. The soap bar according to claim 13 wherein the first and second members are arranged to diverge outwardly from a mid point of the soap bar, and the flat region and/or the further flat region extends from said mid point along the first member and/or the second member.

15. The soap bar of claim 1 wherein the angle of divergence is approximately 145°.

16. The soap bar of claim 1 configured substantially as a boomerang shape.

17. The soap bar of claim 16 wherein at least one face of the soap bar is convex.

18. The soap bar of claim 17 wherein both faces of the soap bar are convex.
